(19) United States
(12) Patent Application Publication
(10) Pub. No.: US 2024/0366937 A1
(43) Pub. Date: Nov. 7, 2024

(54) DEVICES AND METHODS FOR TREATING TESTICULAR PAIN

(71) Applicant: PathRight Medical Inc., Wayzata, MN (US)

(72) Inventor: Landon Trost, Orem, UT (US)

(21) Appl. No.: 18/589,921

(22) Filed: Feb. 28, 2024

Related U.S. Application Data
(60) Provisional application No. 63/463,138, filed on May 1, 2023.

(51) Int. Cl. A61N 1/36; A61N 1/04

(52) U.S. Cl. CPC A61N 1/36021; A61N 1/0476; A61N 1/0484; A61N 1/0492; A61N 1/36034

(57) ABSTRACT

The present disclosure relates to a method of treating testicular pain. The method includes externally coupling an electrical energy source to a pelvic region of a subject, and delivering electrical energy to one or more areas of the pelvic region.

### CROSS-REFERENCE TO RELATED APPLICATION

The priority benefit of U.S. Provisional Application 63/463,138, filed May 1, 2023, is hereby claimed and the entire contents are incorporated by reference.

### TECHNICAL FIELD

The present disclosure relates to devices and methods for treating testicular pain, and in particular, to devices and methods that deliver electrical energy to a pelvic region to treat testicular pain.

### BACKGROUND

Testicular pain may be caused by a variety of injuries or disorders in men. For example, iliohypogastric neuralgia, ilioinguinal neuralgia, genitofemoral neuralgia, chronic groin pain, chronic testicular pain (CTP), post vasectomy pain, hypogastric nerve injury, anterior extrusions, and other pain originating from the testicles, groin, or abdomen are common reasons for referral to a urological specialist. The testicular pain experienced by a subject may be unilateral or bilateral, constant or intermittent, spontaneous or exacerbated by physical activities and pressure. Pain may remain localized in the scrotum or radiate to throughout the pelvic region to the groin or perineum or extend to the back or legs.

### SUMMARY

According to the present disclosure, devices and methods for non-invasively treating testicular pain may be adapted to a variety of physiologies of male subjects to treat pain points generally in the pelvic region, and specifically near, above, or on the scrotum.

In a first example aspect, a method of treating testicular pain may include externally coupling an electrical energy source to a pelvic region of a subject, and delivering electrical energy to one or more areas of the pelvic region.

In a second example aspect, a method of treating testicular pain in a subject may include coupling a stimulation device to a scrotum of the subject. The stimulation device may include an electrical energy generator and a contact coupled to the generator and configured for delivering electrical energy. The method may include delivering, via the stimulation device, electrical energy to the subject.

In a third example aspect, a wearable device for treating testicular pain may include a stimulation contact configured for delivering energy to a pelvic region of a subject. A generator may be coupled to the stimulation contact and may be configured for transmitting energy to the stimulation contact.

In accordance with any one of the first, second, and third example aspects, a method for treating testicular pain and a wearable device for treating testicular pain may include any one or more of the following forms.

In one form, externally coupling an electrical energy source may include engaging the electrical energy source with a scrotum of the subject.

In some forms, externally coupling the electrical energy source may include coupling an electrical stimulation device to a scrotum of the subject.

In other forms, wherein the electrical energy source may include a first electrode and a second electrode of the electrical stimulation device.

In another form, coupling the electrical stimulation device may include attaching a cuff at least partially around the scrotum of the subject.

In some forms, the cuff may include an electrode positioned to engage the scrotum when the cuff is attached to the scrotum.

In yet another form, externally coupling the electrical energy source may include coupling an electrode of an electrical stimulation device to a scrotum of the subject.

In one example, externally coupling the electrical energy source may include coupling a second electrode to a different area of the pelvic region.

In some examples, externally coupling the electrical energy source may include adhering an electrode to a pelvic region adjacent to an inguinal canal and/or cremasteric muscle.

In other examples, delivering the electrical energy may include delivering an electrical pulse to the inguinal canal and/or cremasteric muscle of the subject.

In yet another example, delivering the electrical energy may include delivering an electrical pulse to a testicular cord, a cremasteric muscle, an inguinal canal, a genitofemoral nerve, an ilioinguinal nerve, and/or an iliohypogastric nerve of the subject.

In one aspect, the method may include adjusting a frequency of the electrical pulse being delivered.

In some aspects, the frequency may be in a range of approximately 0 Hz to approximately 150 Hz.

In some aspects, the method may include adjusting a pulse width of the electrical pulse being delivered.

In one aspect, the pulse width may be in a range of approximately 50 microseconds to approximately 200 microseconds.

In other aspects, externally coupling may include coupling a first electrode of an electrical stimulation device to a first area of the pelvic region and coupling a second electrode of the electrical stimulation device to a second area of the pelvic region.

In another aspect, coupling the stimulation device may include positioning a second contact in proximity to an inguinal canal of the subject.

In yet another aspect, coupling the stimulation device may include positioning a second contact of the stimulation device in an area different than the scrotum in a pelvic region of the subject.

In one form, the method may include positioning a cuff of the stimulation device around the scrotum of the subject.

In other forms, the contact may be integrated with the cuff of the stimulation device.

In some forms, delivering electrical energy may include delivering an electrical pulse to the subject.

In other forms, the method may include adjusting a setting of the electrical pulse.

In another form, adjusting the setting may include communicating with a controller using a remote communication device.

In some forms, the controller may be communicatively coupled to the electrical energy generator.

In yet another form, the method may include adjusting a frequency of the one or more electrical pulses being delivered.

In one example, the frequency may be in a range of approximately 0 Hz to approximately 150 Hz.

In one example, the method may include adjusting a pulse width of the electrical pulse being delivered.

In some examples, the pulse width may be in a range of approximately 50 microseconds to 200 microseconds.

In some examples, delivering the electrical energy may include delivering an electrical pulse to an inguinal canal of the subject.

In other examples, delivering the electrical energy may include delivering an electrical pulse to a genitofemoral nerve of the subject.

In yet another example, delivering the electrical energy may include delivering an electrical pulse to an ilioinguinal nerve of the subject.

In one aspect, delivering the electrical energy may include delivering an electrical pulse to an iliohypogastric nerve of the subject.

In some aspects, delivering the electrical energy may include delivering an electrical pulse to a cremasteric muscle of a testicular cord.

In other aspects, the device may include a garment defining a pocket sized to receive the generator.

In some aspects, the generator may be insertable in the pocket of the garment.

In another aspect, the stimulation contact may include an electrode configured for delivering an electrical pulse.

In yet another aspect, the device may include a flexible band arranged for removably attaching to a scrotum of a subject.

In one form, the electrical stimulation source may be coupled to the band.

In one form, the band may be arranged to form a circular cuff sized to fit around a scrotum of a subject.

In some forms, a controller may be operatively coupled to the generator.

In another form, the controller may include a processor and a memory communicatively coupled to the processor and storing executable instructions that, when executed by the processor, causes the processor to receive data transmitted by a remote communication device, and send a signal to the generator.

In other forms, the instructions may cause the processor to adjust an energy parameter of the energy generated by the generator.

### Definitions

As used herein, the terms “top,” “bottom,” “upper,” “lower,” “above,” and “below” are used to provide a relative relationship between structures. The use of these terms does not indicate or require that a particular structure must be located at a particular location in the apparatus.

Some examples may be described using the expression “coupled” and “connected” along with their derivatives. For example, some arrangements may be described using the term “coupled” to indicate that two or more elements are in direct physical or electrical contact. The term “coupled,” however, may also mean that two or more elements are not in direct contact with each other, but yet still co-operate or interact with each other. The examples described herein are not limited in this context.

As used herein, the term “pelvic region” or “pelvic/scrotal region” may be used to refer to right, left, or both sides of one or more of a lower pelvic region, scrotum, perineum, epididymis, vas deferens, testicle, testicular cord (or spermatic cord), cremasteric muscle(s), inguinal ring, inguinal canal, genitofemoral nerve, ilioinguinal nerve, iliohypogastric nerve, and pudendal nerve.

Examples of the present disclosure can include one or more of the following advantages.

The disclosed methods and devices may advantageously increase the signal coming into the targeted areas (e.g., the testicular cord, including the testicular cremasteric muscles, inguinal canal, ilioinguinal nerve, the iliohypogastric nerve, and/or iliohypogastric nerve) and downregulate the sensitivity of the spinal cord/brain of the subject, such that the normal pain signals coming in will no longer be sufficient to register as pain.

In some examples, the disclosed methods and devices may be used to treat scrotal pain, chronic pelvic pain, meralgia paraesthetica, and cramping discomfort of irritable bowel syndrome.

In some examples, the disclosed methods and devices may provide alternatives to pharmacologic and surgical therapies.

In some examples, the disclosed devices may be integrated with a wearable garment or accessory to provide a discreet device that is low profile and quiet.

In some examples, the disclosed methods and devices may exhaust the ability of the targeted nerves to fire, which can occur with repeated stimulation.

In some examples, the disclosed methods and stimulation devices may be adapted to treat a patient population having subjects in a wide range of weight classes and with scrotal lengths and sizes.

In some examples, the wearable stimulation device of the present disclosure has an integrated, adjustable coupler that permits attaching to a scrotum according to the subject's scrotal physiology or comfort. The adjustable coupler can be tightened or loosened easily.

In another example, the stimulation device with a flexible electrode contact pad may be placed under a subject's panniculus or other areas of the body to effectively treat testicular pain. The electrodes are flexible and come in a variety of shapes and sizes to best suit a subject's physiology and comfort.

Other features and advantages of the present disclosure will be apparent from the following detailed description, figures, and claims.

### BRIEF DESCRIPTION OF THE DRAWINGS

FIG. 1 is a front view of a first example stimulation device for treating testicular pain in accordance with the teachings of the present disclosure;

FIG. 2 is a front view of the stimulation device of FIG. 1 coupled to a pelvic region of a human subject;

FIG. 3 is a flow diagram of an example method of treating testicular pain in a subject in accordance with the teachings of the present disclosure;

FIG. 4 is a back view of a second example stimulation device for treating testicular pain assembled in accordance with the teachings of the present disclosure;

FIG. 5 is a front view of the stimulation device of FIG. 4;

FIG. 6 is a front view of the stimulation device of FIG. 4 shaped to form a circular cuff;

FIG. 7 is a front view of the stimulation device of FIG. 4 coupled to a pelvic region of a human subject;

FIG. 8 is a front view of a wearable assembly for treating testicular pain in accordance with the teachings of the present disclosure;

FIG. 9 is a front view of a wearable system for treating testicular pain assembled in accordance with the teachings of the present disclosure; and

FIG. 10 is a schematic drawing of a control system that can be used to perform operations in accordance with the teachings of the present disclosure.

### DETAILED DESCRIPTION

According to the present disclosure, devices and methods for non-invasively treating testicular pain may be adapted to a wide variety of physiologies of subjects to treat pain in the pelvic region, and specifically, near, above, and/or on the scrotum.

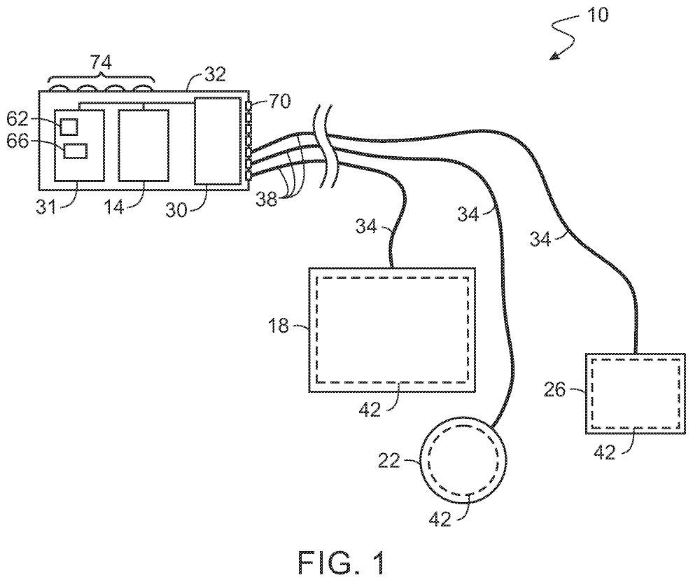

In FIG. 1, a first example stimulation device 10 is configured for external stimulation of a pelvic region of a subject. The electrical stimulation device 10 includes an electrical signal generator 14, a plurality of electrical contacts 18, 22, 26 coupled to the electrical signal generator 14, and a power source 30 coupled to the generator 14. The generator 14, power source 30, and a controller 31 are disposed in a generator housing 32.

The stimulation device 10 includes first 18, second 22, and third 26 electrical contacts, and each contact 18, 22, 26 includes a wire 34 having a plug 38 at a first end and a contact pad 42, which delivers the electrical energy to the subject, at an opposing end. Each wire 34 electrically couples the contact 18, 22, 26 to the generator 14. The contacts 18, 22, 26 are arranged for placement at various areas on or near a pelvic region of a subject to deliver stimulating energy to the subject to treat testicular pain.

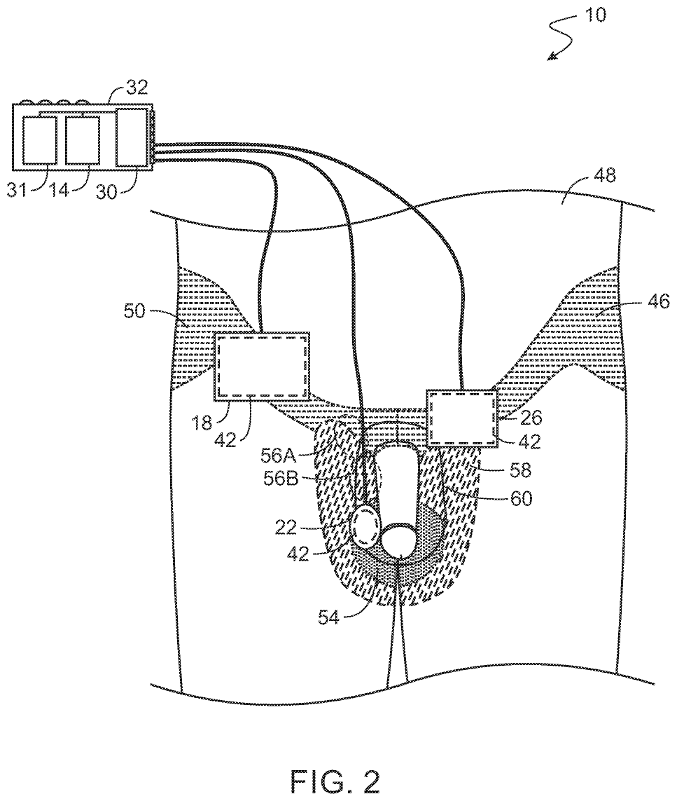

As shown in FIG. 2, the contacts 18, 22, 26 are placed at various locations on a pelvic region 46 of a subject 48. The contact pad 42 of the first contact 18 is coupled to a first area 50 of the subject's pelvic region 46, and specifically adjacent to the inguinal canal of the subject's right side to stimulate an iliohypogastric nerve. The contact pad 42 of the second contact 22 is coupled to a second area 54 of the subject's pelvic region 46, specifically on a right testicle of the scrotum 60 to stimulate a genitofemoral nerve of the subject 48. And the contact pad 42 of the third contact 26 is coupled to a third area 58 of the subject's pelvic region 46, and specifically adjacent to an inguinal canal of the subject's lower left side to stimulate both an ilioinguinal nerve and the iliohypogastric nerve.

The contacts 18, 22, 26 may be coupled to the subject at a combination of locations to best stimulate the testicular cord, including the testicular cremasteric muscles, inguinal canal, ilioinguinal nerve, the iliohypogastric nerve, and/or iliohypogastric nerve of the subject. In FIG. 2, first and second dashed areas 56A, 56B represent additional areas for placing one or more contacts on the scrotum 60 to treat testicular pain. It will be appreciated that the contacts 18, 22, 26 may be attached to any area of the pelvic region 46, such as, for example, along a circumference and length of the scrotum 60.

In the illustrated example of FIGS. 1 and 2, the first contact 18 has a large rectangular electrode pad 42, the second contact 22 has a circular electrode pad 42, and the third contact 26 has a small rectangular electrode pad 42. The contacts may be selected from a variety of contacts of various sizes and shapes depending on the treatment area and physiology of the patient. Once selected, the plug 38 of each contact may then be coupled to a socket 70 of the generator housing 32 before initiating treatment. Each contact pad 42 of the contacts 18, 22, 26 has a contact surface that is configured to adhere to a skin surface of the subject 48. The contact surface may include a textured surface, adhesive, or other material that is configured for self-adhering or otherwise attaching to a skin surface securely (e.g., by suction or friction). Additionally, the contact surface may be removed and reattached to a skin surface repeatedly.

While the stimulation device 10 of FIGS. 1 and 2 includes three contacts 18, 22, 26, each having a different shape and size, other configurations of the stimulation device 10 are possible. In some examples, the contacts may be the same size and shape, and may be a different shape than depicted in FIGS. 1 and 2. For example, to treat a wide area, one or more of the contacts may be long rectangles that extend partially or entirely across a subject's pelvis. In other examples, the contacts may be combined to create a variety of different geometries.

Turning back to FIG. 1, the generator housing 32 of the device 10 houses the generator 14, power source 30, and control system 31, and defines a plurality of sockets 70 and a plurality of buttons 74. The control system 31 can communicate through signals (e.g., wired or wirelessly) to one or more components of the device 10. The controller 31 includes a processor 62 and a memory 66 that stores executable instructions that, when executed by the processor 62, can control certain settings and energy parameters of the stimulation device 10. For example, energy parameters may be adjusted to provide a deliverable electrical energy having a desired intensity, frequency, amplitude, and duration. In some aspects, control system 31 is a micro-processor based control system (or controller) that includes, e.g., hardware processor(s), memory module(s), and instructions executable as software code to cause the processor(s) to perform operations to control one or more components of the device 10. However, the control system 31 can also be realized as a mechanical, electro-mechanical, hydraulic, pneumatic, or other form of a control system or controller without departing from the scope of this disclosure.

Each of the wires 34 of the contacts 18, 22, 26 is removably coupled to a socket 70 formed in the generator housing 32. So configured, a subject can customize the stimulation device 10 by attaching a suitable contact of size and shape, and number of contacts. A plurality of sockets 70 are available for receiving additional contacts if more than three contacts are desired for treatment. The buttons 74 partially extend outside of the housing 32 for user interaction and are configured to transmit a variety of different signals to the controller 31 to operate the stimulation device 10. For example, one or more buttons 74 may be pressed to turn on/off the device 10, increase or decrease an energy parameter (e.g., pulse width, intensity, frequency, amplitude), or to adjust or program various settings of the stimulation device 10 (e.g., duration of delivering electrical energy, setting alarms, providing modes, or programming treatment plans). In some examples, one or more dials, knobs, switches, may be used with or instead of the buttons 74 to operate the device 10.

The contacts 18, 22, 26 are configured to deliver electrical energy to the subject 48 via electrical pulses. The pulses can be adjusted by the subject or healthcare provider to a desired frequency, pulse width, amplitude, duration (i.e., time of the entire treatment), and mode (e.g., burst, continuous, other pattern) in accordance with a treatment plan and/or a comfort level of the subject 48. For example, the frequency, or the number of electrical pulses delivered in one second, may be selected from a range of approximately 10 Hz or more (e.g., about 15 Hz or more, about 20 Hz or more, about 25 Hz or more, about 30 Hz or more, about 35 Hz or more, about 40 Hz or more, or about 45 Hz) to approximately 100 Hz or less (e.g., about 95 Hz or less, about 90 Hz or less, about 85 Hz or less, about 80 Hz or less, about 75 Hz or less, about 70 Hz or less, about 65 Hz or less, about 60 Hz or less, about 55 Hz or less, about 50 Hz or less, or about 45 Hz). A pulse width, or the elapsed time of a single pulse of energy, may be selected from a range of approximately 50 μs or more (e.g., about 60 μs or more, about 70 μs or more, about 80 μs or more, about 90 μs or more, about 100 μs or more, about 110 μs or more, about 120 μs or more, about 130 μs or more, about 140 μs or more, about 150 μs or more, about 160 μs or more, about 170 μs or more, about 180 μs or more, about 190 μs or more, or about 200 μs) to approximately 400 μs or less (e.g., about 390 μs or less, about 380 μs or less, about 370 μs or less, about 360 μs or less, about 350 μs or less, about 340 μs or less, about 330 μs or less, about 320 μs or less, about 310 μs or less, about 300 μs or less, about 290 μs or less, about 280 μs or less, about 270 μs or less, about 260 μs or less, about 250 μs or less, about 240 μs or less, about 230 μs or less, about 220 μs or less, about 210 μs or less, or about 200 μs). An amplitude of the pulse may be selected from a range of approximately 1 mA or more (e.g., about 5 mA or more, about 10 mA or more, about 15 mA or more, about 20 mA or more, about 25 mA or more, about 30 mA or more, about 35 mA or more, about 40 mA or more, about 45 mA or more, or about 50 mA) to approximately 100 mA or less (e.g. about 95 mA or less, about 90 mA or less, about 85 mA or less, about 80 mA or less, about 75 mA or less, about 70 mA or less, about 65 mA or less, about 60 mA or less, about 55 mA or less, or about 50 mA).

An example treatment plan for one or more electrical contacts may include delivering a first electrical pulse at a lower frequency for a period of time, and gradually or periodically, increasing the frequency. Another example treatment may include applying intermittent pulses so that the electrical contact delivers pulses at a first frequency for a first time period, and stops for a second time period, and delivers electrical pulses at the first or different frequency for a third time period after rest. Another example treatment may be fully customizable by the subject. These treatment plans may be done in unison or independently from the other electrical contacts.

In other examples, the generator housing 32 may include a user interface (e.g., a display screen, touchscreen, etc.) instead of or in addition to buttons 74 for operating and/or adjusting the energy parameters and settings of the stimulation device 10. As will be described in detail below, a subject 48 may adjust the energy parameters and settings of the stimulation device 10 through a personal electronic device, such as a smartphone, smartwatch, laptop, or other remote computer that can communicate wirelessly (e.g., via Bluetooth) with the controller 31 housed in the generator housing 32.

To treat testicular pain in the subject 48, a healthcare provider may first identify a location of testicular pain. After identifying the location of pain, the healthcare provider can recommend a target treatment area or location on the pelvic region 46 to treat the identified location of testicular pain. In some examples, the area or location of pain may be in a different area or location than the target treatment area or region. Generally speaking, the subject 48, caregiver, or other healthcare provider can apply the contacts 18, 22, 26 to various areas of the pelvic region 46 by adhering the pads 42 to the subject's skin. Once attached, the subject 48 or caregiver can deliver electrical energy to the pelvic region 46 through the contacts 18, 22, 26 by operating the device 10. The subject 48 or caregiver can adjust the energy parameters of the deliverable electrical energy to operate and manage an electrical stimulation treatment plan using the electrical stimulation device 10. The stimulation device 10 can operate for a predetermined duration, e.g., 5 minutes to 60 minutes, or can operate in a single delivery of an electrical pulse or shock to the target areas.

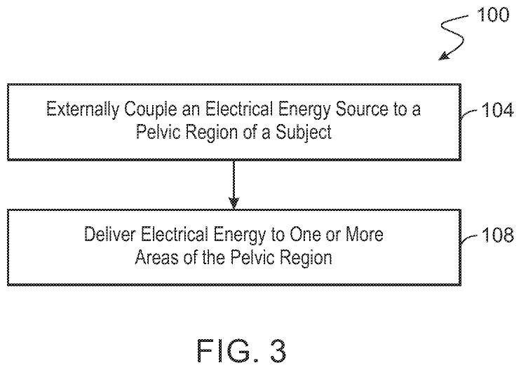

Turning now to FIG. 3, a flow chart represents an example method 100 for treating testicular pain using electrical stimulation in accordance with the teachings of the present disclosure. The example method 100 illustrated in FIG. 3 may be performed with the stimulation device 10 of FIGS. 1 and 2 or using another device. A step 104 of the method 100 includes externally coupling an electrical energy source to a pelvic region 46 of a subject 48. Externally coupling the electrical energy source using the device 10 of FIGS. 1 and 2, for example, includes attaching each contact 18, 22, 26 to the subject 48. Once attached, the method 100 includes a step 108 of delivering energy to one or more areas of the pelvic region 46. For example, using the device 10 of FIGS. 1 and 2, electrical energy is delivered to the subject 48 through multiple electrical pulses through the contacts 18, 22, 26.

The electrical energy source may be part of an electrical stimulation device, such as the device 10 of FIGS. 1 and 2, or a different example device that generates and/or transmits electrical energy. In one example, externally coupling an electrical energy source includes engaging an electrode with a skin surface of the subject 48. In other examples, this step 104 may include attaching a first electrode to a first area of the pelvic region 46 and attaching a second electrode to a second area of the pelvic region 46. In some examples, step 104 may include adhering a first electrode to a region adjacent to an inguinal nerve or canal of the subject 48. In some examples, step 104 may include coupling an electrode to a scrotum 60 of the subject 48. In yet other examples, the energy source may be something other than an electrode, such as a defibrillator or device or material that permits electricity to enter or leave an object, substance, or region.

The step 108 of delivering energy to one or more areas of the pelvic region 46 may include delivering an electrical pulse to the inguinal canal, testicular cord, including the testicular cremasteric muscles, a genitofemoral nerve, an ilioinguinal nerve, and/or an iliohypogastric nerve of the subject 48. Delivering electrical energy may include delivering multiple electrical pulses over a period of time or delivering a single electrical shock to one or more areas of the pelvic region 46. In some examples, delivering multiple electrical pulses may include constant stimulation, infrequent bursts, or a combination of modes and methods.

The method 100 may include other steps for operating and controlling energy delivery to the subject's pelvic region 46. In some examples, the method 100 may include communicating with a controller 31 to execute instructions stored on a memory 66 of the controller 31 to adjust certain device settings or energy parameters of the deliverable electrical energy. Before or after delivering the electrical energy to the subject 48, the subject or caregiver may adjust a frequency, pulse width, and/or amplitude of the electrical pulses being delivered. Alternatively, these and other settings may be set before delivering any stimulating treatment to the subject 48.

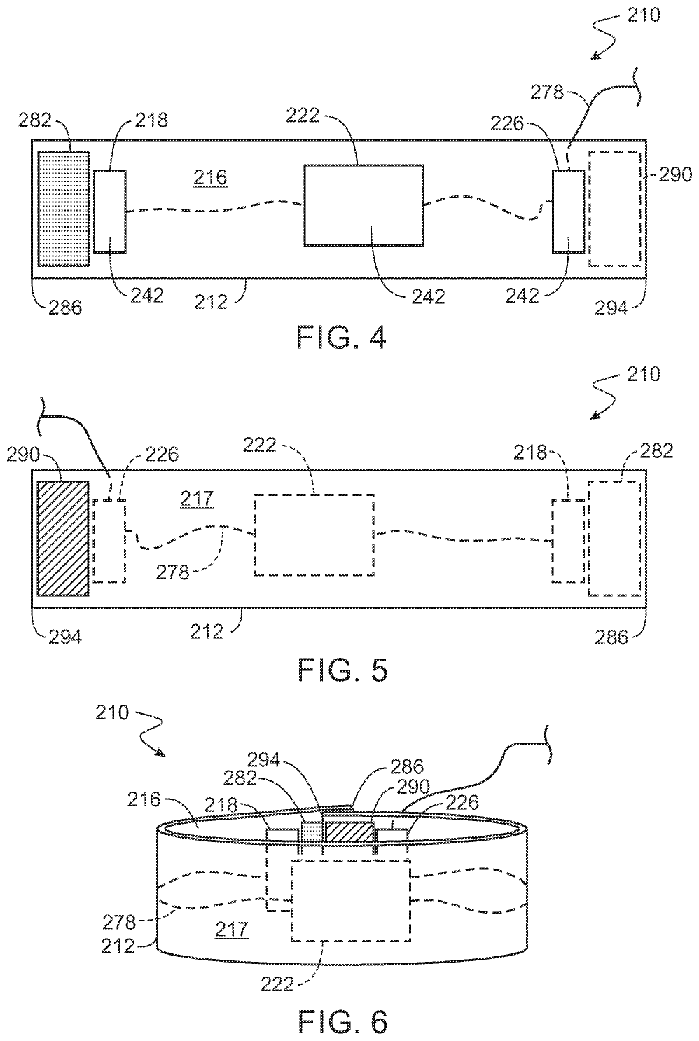

In FIGS. 4-7, a second example electrical stimulation device 210 includes an electrical energy source for delivering electrical energy to a pelvic region 46 of a subject 48, and for performing the method 100 of FIG. 3. The second example stimulation device 210 is similar to the first example stimulation device of FIGS. 1 and 2, and the electrical energy source includes three electrical contacts 218, 222, 226 for delivering electrical energy to a subject 48. Thus, for case of reference, and to the extent possible, the same or similar components of the second example device 210 will retain the same reference numbers as outlined above with respect to the first example device 10, although the reference numbers will be increased by 200. However, the second example device 210 differs from the first example device 10 in the manner discussed below.

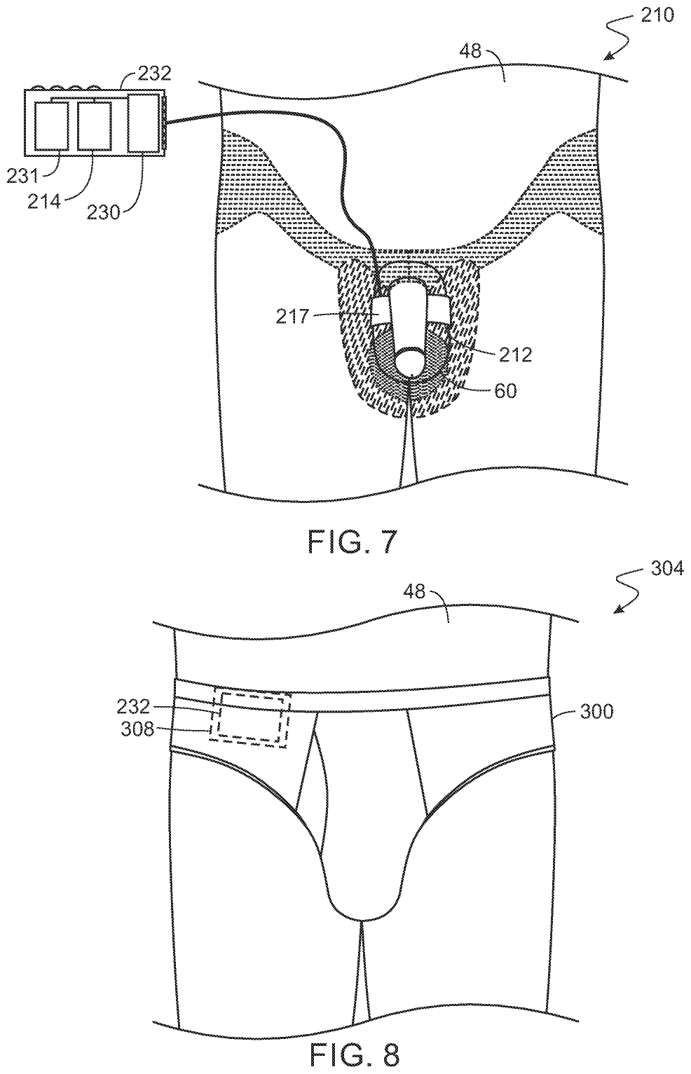

The second example device 210 includes a flexible body 212 including an interior surface 216 (FIG. 4), an exterior surface 217 (FIG. 5), three electrodes 218, 222, 226 attached to (e.g., embedded between) one or more layers of the body 212, and a generator housing 232 that houses an electrical signal generator 214, battery 230, and control system 231 (FIG. 7). In FIG. 4, each contact pad 242 of the electrodes 218, 222, 226 is exposed on the interior surface 216 of the body 212, and in FIG. 5, the electrodes 218, 222, 226 are covered by a layer of material on the exterior surface 217. As shown in FIG. 6, the body 212 can be arranged into a cuff that is sized to fit at least partially around a scrotum 60 of the subject 48. The cuff is arranged so that the interior surface 216 engages the scrotum 60 of the subject 48, as shown in FIG. 7. In this way, the cuff can stimulate the testicular cord and cremasteric muscle of the subject 48 to treat testicular pain.

The electrodes 218, 222, 226 are spaced relative to each other in a way that when the cuff is positioned on the scrotum 60, the contact pads 242 of the electrodes 218, 222, 226 engage different areas of the subject's scrotum 60. When arranged on the scrotum 60, the first and third electrodes 218, 226 are placed on a back side of the scrotum 60, and the second electrode 222 is placed on a front side of the scrotum 60. However, the cuff may be rotated to stimulate different areas of the scrotum 60. In other examples, the body 212 may include additional electrodes placed variously along a length of the body 212. The electrodes 218, 222, 226 are electrically coupled by a wire 278 embedded between one or more layers of the body 212. The wire 278 extends externally from the body 212 to connect with a generator housing 232 (FIG. 7). The device 210 may be operated in the same or similar manner as the device 10 by adjusting the settings of the device 210 and/or energy parameters of the deliverable energy using the buttons 274 and/or the control system 231.

The device 210 includes an adjustable coupler to treat a range of scrotal sizes. The coupler includes a first coupling 282 disposed at a first end 286 of the body 212 and a second coupling 290 disposed at the second end 294 of the body 212. The first and second couplings 282, 290 are disposed on opposite surfaces 216, 217 of the body 212 such that when the first and second ends 286, 294 of the body 212 are joined to form the cuff, the first and second couplings 282, 290 overlap and mate. In the illustrated example, the coupler is a hook-and-loop fastener where the first coupling 282 includes a plurality of hooks that are arranged to fasten to a plurality of loops of the second coupling 290.

In some examples, the body 212 of the second example stimulation device 210 may have a microcontroller, power source, and/or generator integrated into one or more layers of the body 212. In this example, the body 212 could be rechargeable and is configured to directly communicate with a user's mobile electronic device for operation.

In some examples, the second example device 210 may include more or fewer electrodes that may be activated or deactivated depending on the treatment plan to treat testicular pain.

In some examples, the body 212 of the second example stimulation device 210 may be arranged differently so that the cuff is formed using, for example, a tie, buckle, button, snap, or other suitable male-female mating structure or fastener. In other examples, the body 212 is circular and only needs slight adjustment after placing the device 210 around the scrotum 60 of the subject. In this case, rather than coupling opposing ends of a rectangular strip embedded with electrodes, one example device may be preconfigured as a cuff to receive the scrotum into a central opening of the cuff. In some examples, the body 212 may be made of a memory-shape material that may hold a new shape in response to being manipulated and shaped (e.g., bent, twisted, etc.).

In FIG. 8, a wearable assembly 304 includes a garment 300, which in this example is underwear, and the stimulation device (hidden from view), such as the devices 10, 210. The wearable assembly 304 may be worn discreetly and comfortably by the subject 48. The undergarment 300 has a built-in pocket 308 sized to receive the generator housing 32, 232. The garment 300 fits over the stimulation device 10, 210 attached to the subject 48 so that the subject 48 may wear and easily operate the stimulation device 10, 210 throughout the course of a day.

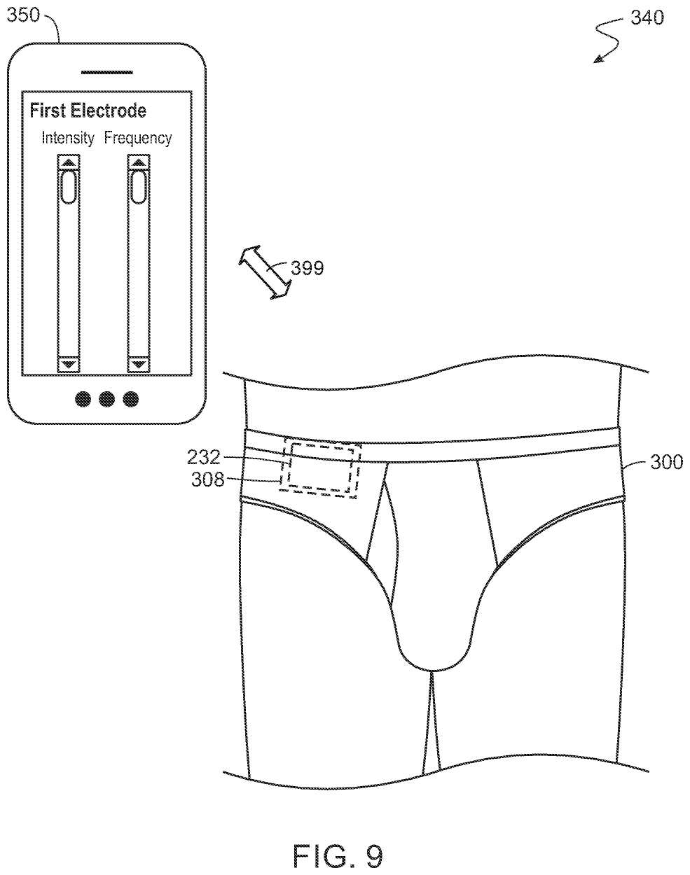

In another example, each of the devices 10, 210 may be operated using a personal electronic device 350, such as a smartphone, smartwatch, laptop, or other remote computer, as shown in an example treatment system 340 of FIG. 9. The treatment system 340 of FIG. 9 includes a stimulation device (hidden from view), such as the stimulation device 10, 210 of FIGS. 1-7, and an application stored on a personal electronic device 350. Using wireless communication, such as via Wi-Fi, the subject 48 can control the device 10, 210 remotely via wireless communication signals 399 rather than retrieving the generator housing 232 from the pocket 308 of the garment 300 and operating the device 10, 210 manually. In this example, the subject 48 can use an application stored on the personal electronic device 350 to operate the stimulation device 10, 210. For example, the subject 48 can adjust the settings and parameters of the stimulation device 10, 210 to deliver a particular stimulating treatment to the pelvic region 46. The application may also be programmable so that the subject can create and store a treatment plan for repeated use. The application may also be remotely accessed by a health care provider so that the health care provider can adjust the settings of the device 10, 210, create, and/or modify treatment programs customized for the subject 48.

In the illustrated example of FIG. 9, the electronic device 350 displays two energy parameters for controlling an energy output of a first electrode. However, in other examples, each electrode may have more or fewer adjustable parameters. Additionally, in some examples, the energy parameters of the electrodes may be adjusted collectively.

In some examples, the controller 31, 231 of the stimulation devices 10, 210 may be configured to selectively adjust the device parameters for each contact. For example, if a first area of the pelvic region requires more intense treatment than a second treated area, the user may adjust the settings of the electrodes so that the first electrode delivers an intense stimulation treatment to the first region and the second electrode delivers a less intense stimulation treatment.

In some examples, the method 100 of treating testicular pain by delivering energy to a subject 48 and the devices 10, 210 may include one or more of the following methodologies for delivery energy: transcutaneous electrical nerve stimulation, Russian stimulation, neuromuscular electrical stimulation (NMES), inferential current (IFC) electrical stimulation, high voltage electrical stimulation, iontophoresis, or a combination thereof.

In some examples, the stimulation devices 10, 210 may be configured for delivering electrical, radiation, magnetic, conducted energy, or a combination thereof to effectively treat testicular pain.

In some examples, the electrical stimulation device 10, 210 may be incorporated into a garment, such as underwear. For example, the underwear pouch that covers a subject's penis and scrotum may include a plurality of spaced electrodes that may be selectively operated to deliver electrical stimulation to various locations of the pelvic region. Other regions of the underwear may also incorporate electrical energy sources.

With reference to FIGS. 1-9, an example operation of the stimulation device 10 can be performed to treat testicular pain. One or more steps of the example operation may be performed by or with the control system 31, 231 (e.g., with input from a human operator). For instance, the operation may include adjusting a pulse intensity, setting a timer for treatment, sending results to a healthcare provider, etc.

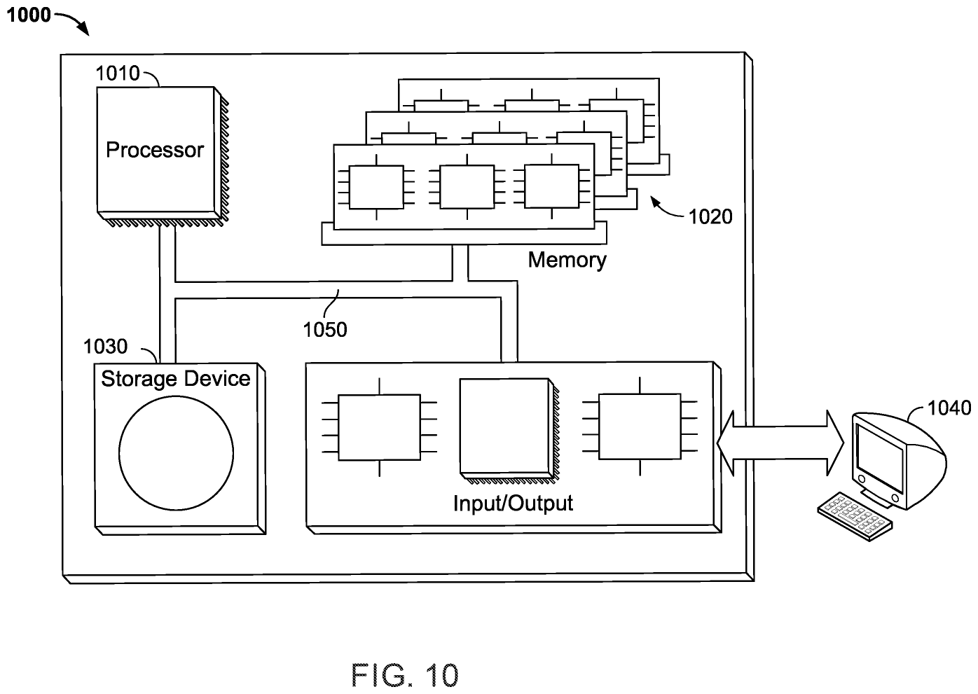

FIG. 10 shows a schematic drawing of a control system 1000 that can be used, e.g., as control system 31, 231, to perform operations according to the present disclosure. Some or all of the example control system 1000 can be implemented as cloud-based system and/or service, alone or in combination with other portions of the example control system 1000 that can be implemented to execute. The controller 1000 is intended to include various forms of digital computers, such as printed circuit boards (PCB), processors, digital circuitry, or otherwise. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the controller 1000. The processor may be designed using any of a number of architectures. For example, the processor 1010 may be a CISC (Complex Instruction Set Computers) processor, a RISC (Reduced Instruction Set Computer) processor, or a MISC (Minimal Instruction Set Computer) processor.

In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the control system 1000. In one implementation, the memory 1020 is a computer-readable medium. In one implementation, the memory 1020 is a volatile memory unit. In another implementation, the memory 1020 is a non-volatile memory unit.

The storage device 1030 is capable of providing mass storage for the controller 1000. In one implementation, the storage device 1030 is a computer-readable medium. In various different implementations, the storage device 1030 may be a floppy disk device, a hard disk device, an optical disk device, a tape device, flash memory, a solid state device (SSD), or a combination thereof.

The input/output device 1040 provides input/output operations for the controller 1000. In one implementation, the input/output device 1040 includes a keyboard and/or pointing device. In another implementation, the input/output device 1040 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, solid state drives (SSDs), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) or LED (light-emitting diode) monitor for displaying information to the user and a keyboard and a pointing device such as a mouse or a trackball by which the user can provide input to the computer. Additionally, such activities can be implemented via touchscreen flat panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any disclosure or of what may be claimed, but rather as descriptions of features that may be specific to particular examples of particular disclosures. Certain features that are described in this specification in the context of separate examples can also be implemented in combination in a single example. Conversely, various features that are described in the context of a single example can also be implemented in multiple examples separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the examples described herein should not be understood as requiring such separation in all examples, and it should be understood that the described program components and systems can generally be integrated together in a single product or packaged into multiple products.

Particular examples of the subject matter have been described. Other examples are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method of treating testicular pain, the method comprising:
externally coupling an electrical energy source to a pelvic region of a subject; and
delivering electrical energy to one or more areas of the pelvic region.

2. The method of claim 1, wherein externally coupling the electrical energy source comprises connecting the electrical energy source with a scrotum of the subject.

3. The method of claim 1, wherein externally coupling the electrical energy source comprises coupling an electrical stimulation device to a scrotum of the subject, wherein the electrical stimulation device comprises an electrode.

4. The method of claim 3, wherein coupling the electrical stimulation device comprises attaching a cuff at least partially around the scrotum of the subject, wherein the cuff comprises the electrode positioned to engage the scrotum when the cuff is attached to the scrotum.

5. The method of claim 3, comprising coupling a second electrode to a different area of the pelvic region.

6. The method of claim 1, wherein externally coupling the electrical energy source comprises adhering an electrode to a region adjacent to an inguinal canal, and wherein delivering the electrical energy comprises delivering an electrical pulse, via the electrode, to the inguinal canal.

7. The method of claim 6, wherein delivering electrical energy comprises delivering an electrical pulse having frequency in a range of approximately 0 Hz to approximately 150 Hz.

8. The method of claim 7, comprising adjusting the frequency of the electrical pulse being delivered.

9. The method of claim 1, wherein delivering the electrical energy comprises delivering an electrical pulse to a testicular cord, a cremasteric muscle, an inguinal canal, a genitofemoral nerve, an ilioinguinal nerve, and/or an iliohypogastric nerve of the subject.

10. A method of treating testicular pain in a subject, the method comprising:
coupling a stimulation device to a scrotum of the subject, the stimulation device comprising an electrical energy generator and a contact configured to couple to the electrical energy generator and to deliver electrical energy; and
delivering, via the stimulation device, electrical energy to the subject.

11. The method of claim 10, wherein coupling the stimulation device comprises positioning a second contact of the stimulation device in an area different than the scrotum in a pelvic region of the subject.

12. The method of claim 10, comprising positioning a cuff of the stimulation device around the scrotum of the subject, wherein the contact is integrated with the cuff of the stimulation device.

13. The method of claim 10, wherein delivering electrical energy comprises delivering an electrical pulse to the subject.

14. The method of claim 13, comprising adjusting a setting of the electrical pulse.

15. The method of claim 14, wherein adjusting the setting comprises communicating with a controller using a remote communication device, wherein the controller is communicatively coupled to the electrical energy generator.

16. A wearable device to treat testicular pain, the device comprising:
a stimulation contact configured for delivering energy to a pelvic region of a subject;
a generator configured to couple to the stimulation contact, the generator configured for transmitting energy to the stimulation contact.

17. The device of claim 16, comprising a garment defining a pocket sized to receive the generator, the generator being insertable in the pocket of the garment.

18. The device of claim 16, wherein the stimulation contact comprises an electrode configured for delivering an electrical pulse.

19. The device of claim 16, comprising a flexible band arranged to removably attach to a scrotum of a subject, wherein the stimulation contact is coupled to the flexible band.

20. The device of claim 16, comprising a controller operatively coupled to the generator, the controller comprising a processor, a memory communicatively coupled to the processor and storing executable instructions that, when executed by the processor, causes the processor to:
receive data transmitted by a remote communication device;
send a signal to the generator; and
adjust an energy parameter of the energy generated by the generator.
